(19) United States
(12) Patent Application Publication
(10) Pub. No.: US 2003/0044808 A1
(43) Pub. Date: Mar. 6, 2003

(54) Method and system of single labeling and parallel analysis of differential gene

(76) Inventor: Shun Luo, Irvine, CA (US)

Correspondence Address: DEVINE, MILLIMET & BRANCH, P.A., 111 AMHERST STREET, BOX 719, MANCHESTER, NH, 03105, US

(21) Appl. No.: 10/020,025

(22) Filed: Dec. 7, 2001

Related U.S. Application Data
(63) Continuation-in-part of application No. 09/943,937, filed on Aug. 31, 2001.

(51) Int. Cl. C12Q 1/68; G06F 19/00; G01N 33/48; G01N 33/50

(52) U.S. Cl. 435/6; 702/20

(57) ABSTRACT

The present invention discloses a process using one, not two, labels to perform parallel analysis of multiple samples for their differential gene expression profiles. This process is achieved by using a platform technology, which integrates current DNA micro array and current high throughput screening technology. The invention defines a process that may use a single label to carry out parallel comparison of multiple gene expression samples. The process takes advantage of high density DNA micro array technology and high throughput automation equipment to perform high throughput gene expression analysis, but uses only a single label. Such labeling and analysis reduces variations found in dual fluorescent dye labeling and converts the current manual sample handling to automated sample processing. Thus, the invention enables the transformation of the current DNA micro array technology into a high throughput screening tool.

This Application is a Continuation-In-Part of application Ser. No. 09/943,937 filed on Aug. 31, 2001, which is herein incorporated in its entirety.

### FIELD OF THE INVENTION

The invention relates generally to micro array technology and high throughput screening. More particularly, the invention relates to the combination of micro array technology and a high throughput platform. Most particularly the system and method enable one to do DNA micro array screening in a high throughput screening format, which will hereinafter be generally referred to as “KnowledgeWell™ bio grid array” or “Bio Grid Array”, “Gene Grid Array”, “Protein Grid Array”, “PNA Grid Array”, “Cell Grid Array” or any other “Grid Array” that can be deposited and used in or with the present invention. The present invention is particularly useful to simultaneously create a series of micro arrays in a grid format, each comprising hundreds or thousands of specific gene sequences in forms of DNA fragments such as oligonucleotides, PCR amplification products, cDNA, or genomic DNA. Such a bio grid array can also be used for protein, peptidalnucleic acid (PNA), carbohydrate, RNA, or other biological and biochemical arrays.

A bio grid array, according to the present invention, made with DNA, protein, PNA, RNA, carbohydrate, or other biological and biochemical materials is defined in the form or footprint of a standard microtiter plate conforming to the standards set by the Society of Biomolecular Screening (SBS), thus combining the two technologies and enabling the screening of much greater numbers of DNA micro arrays than currently possible, using the standard micro titer plate format in a high throughput system.

### BACKGROUND OF THE INVENTION

On Feb. 12, 2001, scientists from government and private sectors published that 99% of human genome project was finished with respect to sequencing and assembly (As the Future Catches You: Juan Enriguez Cabot, 2001). This marked the next giant step forward for human beings since landing on the Moon. Scientists have anticipated that the human genome project sequence based genomics technology would drive a higher level of success in pharmaceutical and biotechnological drug discovery. The hope is to reduce the cost and increase the speed of the drug discovery process, one of the primary goals being to produce tailor-made drugs to result in the practice of personalized medicine.

In pursuing such a goal, scientists and technologists have invented highly automated DNA sequencing technology, highly efficient gene cloning methodology, gene expression analysis tools, and powerful bioinformatics algorisms. Particularly, the DNA chip technology has been a driving force for the high hopes of genomics technology to benefit drug discovery. Furthermore, it has been increasingly clear that gene expression analysis will be the core technology to improve the current drug discovery process in the post genome sequence era.

The current DNA micro array (gene or DNA chip, synonymous) is traditionally made in a microscope slide format. A microscope slide formatted DNA chip can host a high density of specific DNA sequences as defined on 1 to 2 cm square area. See for example U.S. Pat. No. 5,800,992 to Affymetrix and U.S. Pat. No. 6,054,270 and No. 6,150,095 to Southern, and Molecular Cloning: A Laboratory Manual (3 Volumes) Maniatis, T. et al. 1982. Many thousands of different genes can be simultaneously studied for their expression patterns using this format, thus yielding understanding of their biological regulatory mechanisms. However, the form factor of microscope slide based DNA arrays limits processing to a semi-automated procedure at best. The microscope slide based array format is also priced at a premium, which prohibits proper experimental design by limiting the number of conditions to less than those optimally required for a robust experiment. The cumbersome microscope slide format impedes a broader application of DNA chip technology in analyzing gene expression of many biologically relevant samples in a cost-effective manner.

The current DNA chip technology requires at least two labels, usually fluorescent, to analyze differential gene expression of two or more samples. Alternatively, one isotopic labeling requires more than two chips or arrays representing multiple samples. Neither of these approaches is designed to process high volumes of samples, similar to those typically processed in a high throughput drug screening application.

Pharmaceutical and biotechnological drug discovery is mainstreamed at using biologically relevant targets to screen combinatorial numbers of chemical entities that are in the millions. It has been proven successful to use biochemical assays based on specific target molecules such as receptors, enzymes, or modulators to screen millions of chemical entities. These targets are usually used to develop biological or biochemical assays that are taken out of the context of a living cell. Such screening can identify specific agonists or antagonists for a specific biological or biochemical target. In the past 25 years, laboratory automation technology has played a central role in enabling scientist to screen millions of chemical entities in a relatively short period of time.

Laboratory automation technology has evolved over many years. Both optimization and standardization of laboratory robotics have proven fruitful. The cohesive approach to automation in the industry has driven standards, which center around the current microtiter plate format. The standard microtiter plate format is defined by the Society of Biomolecular Screening (SBS). With tens of millions of microtiter plates consumed each year by the drug screening industry, the standards have allowed automation technology to become robust and reliable. However, the current drug screening approach, being isolated away from a living cell environment usually presents results in a lack of specificity of the eventual pharmaceutical compound or results in the isolated compound being unsuitable for pharmacological use. Drug screening scientists hope that genomics technology, lead by DNA chip technology, will expand the perspectives of biochemical assay-based drug screening. Using gene expression profiling for drug screening and validation will improve the biological specificity and selectivity of screened chemical entities in a biologically relevant environment. This is because a gene's expression profile represents an overall response of a living cell to its environmental perturbations.

For example, Viagra™ was screened as a therapeutic using one enzyme target that is relevant to penile muscle contraction. However, Viagra™ is not completely specific to penile muscle. Greater specificity was not possible due to the screening process of using one isolated target, Phosphodiesterase V, which is expressed not just in penis, but also other tissues such as in the cardiovascular system. To gain such penile specificity, more penile factors, or molecules that are both specific to the muscle and to the penile regulation mechanism are required in such screening.

Thus, such multifactor screening would be desirable to maintain the effectiveness of therapeutics and eliminate any adverse effect on other parts of the body.

DNA Micro Array

The current DNA micro array technology is focused on high-density DNA depositions on two major substrates: 1.) glass microscope slide, and 2.) nylon or nitrocellulose membrane.

Currently DNA chips and micro arrays are being used for gene expression analysis, gene discovery, gene mapping, genotyping, and mutation detection including single nucleotide polymorphism (SNP) detection. The range of applications for DNA chips and micro array technology is growing fast and spreading into such areas as clinical diagnostics, food safety testing, and forensic study to name but a few.

The most basic micro arrays are composed of DNA samples immobilized on glass. Usually tens up to hundreds of thousands of DNA fragments are put on to an approximately 1-2 cm square area of glass surface, the glass surface being a microscope slide, treated with various chemicals such as polylysine. In general there are three different kinds of DNA chips/micro arrays. These are: cDNA arrays, arrays constructed using pre-made oligonucleotides, and arrays constructed using in-situ synthesized DNA. Tiny droplets, each containing a different known reagent, usually polynucleotide or polypeptide biopolymers such as known DNA fragments, cDNA (which are relatively long strands of DNA representing pieces of genes) or short oligonucleotides (which are usually about 20-70 bases long), are deposited and immobilized in a regular array on a solid substrate such as a glass microscope slide. This type of micro array is usually fabricated in two major forms. One form is by synthesizing oligonucleotide sequences directly on a solid phase using photolithographic technology such as the VLSIPS™ technology. The other is by depositing DNA fragments in the form of oligonucleotides, PCR amplification products, or plasmid DNA of complementary DNA (cDNA) clones.

The glass substrate is almost in all cases in microscope slide format. The immobilization of DNA samples onto the glass can be via covalent or non-covalent bonding. These DNA slides are used to allow hybridization on the surface of the glass between the immobilized samples and the DNA or RNA being tested. Micro array assays are designed to give qualitative and quantitative genetic information concerning the tested samples. The term “DNA chips” is usually used to refer to the high-density oligonucleotide arrays generated by in-situ methods and the term “DNA micro arrays” is used to refer to the low and medium density cDNA or oligonucleotide arrays generated by micro-spotting DNA samples onto glass and other types of substrates.

The Current DNA Micro Array Process

The array of dried droplets is exposed to a solution containing an unknown, for example complementary DNA (cDNA) fragments pre-labeled with a fluorescent dye. Usually a pair of fluorescent dyes (such as Cy3 and Cy5) is used to label a pair of samples, samples 1 and 2 respectively, in contrast (for example red and green labels), such as normal versus cancerous cells. This pair of samples is used to synthesize cDNA in which process the fluorescent dye is incorporated into the respective samples. For example binding reactions or hybridizations occur wherever there is a match between the complementary sequence nucleic acids immobilized in the array and the cDNA being tested. The same sequence or species of gene transcript (mRNA) will bind to the same matching spot on the micro array. A competitive hybridization takes place. For example, if a gene is expressed in sample 1 and 2, then the spot on the micro array corresponding to that gene will bind both samples 1 and 2 and will appear yellow. If a gene is expressed only in sample 1, then the spot on the micro array where that sample bound will only appear red. Similarly if a gene is expressed only in sample 2, then the spot on the micro array where that sample bound will only appear green.

The sample with higher expression of the corresponding gene will be represented by a higher intensity of the specific fluorescent dye, Cy3 or Cy5, red or green, etc. Such differential intensity represents the level of the gene expression under the contrast conditions, such as normal versus cancerous cells. Subsequent optical or radiosensitive scanning determines such intensity difference. Usually one sample is a known control and the second is the test or unknown sample. A control is needed for each array, each assay on each microscope slide.

The choice of tag, for example fluorescent dye, used in the micro array procedures is largely determined by the format of the micro array and the instrumentation that is used to detect the fluorescence generated. Because pairs of samples must be used, and two different labels used, problems of consistency in labeling arise. In addition, the number of samples able to be screened at one time is limited because the samples must be paired when using two labels—there must be a control and a test sample for each assay and each slide.

As noted above, the arrays are typically deposited on a solid substrate, commonly a glass microscope slide. Thus, the number of arrays per slide is limited by the size of a common microscope slide, thus leading to the 1-2 cm square area in which arrays are deposited. Thus the size and dimensions of the microscope slide necessarily limit the dimension and density of an array deposited thereon. In addition, it has proven difficult to handle a high volume of such glass microscope slides for parallel processing of multiple samples. The microscope slide format of the current DNA micro array is not compatible with current laboratory automation platforms. Enormous efforts have been made to re-invent a new automation system to process microscope slide-based DNA micro arrays. Such effort has been put into automation compatible with a microscope slide because the glass of a microscope slide is preferred due to the fact that many of the micro array assays are fluorescence assays using very small amounts of the compounds, and the low background fluorescence of glass is needed. In other words, glass is needed because glass offers the highest optical clarity.

Thus, while it is of great value to study tens of thousands of genes involved in any complex biological process and regulation, and gain tremendous insights into any understandings of biological and pathological occurrences, it is challenging to screen large numbers of biological, physiological, and pathological conditions simultaneously with the current microscope slide-based DNA micro array.

Since the introduction of the KnowledgeWell™ bio grid array of Applicant's U.S. patent application for High Throughput Screening Micro Array Platform, of which the present Application is a Continuation-in-Part, and which is incorporated herein by reference, the current laboratory automation platform can be applied to process micro array assays.

Thus, it would be desirable to be able to have a simpler, less-time consuming, less error-prone and more cost-effective, single label, automation-friendly method and system for screening thousands (or more) genes simultaneously. Such a system would have the advantages of both the DNA micro array technique and the advantages of high throughput screening technology.

### SUMMARY OF THE INVENTION

The present invention includes a process of analyzing multiple samples using a DNA micro array platform that provides a focused number of target genes that are specific to pathways of interest in high throughput drug screening. The process relies on the microtiter plate format platform of the previously described invention, as disclosed and claimed in Applicant's prior application for High Throughput Screening Micro Array Platform which enables utilization of multifactor screening capability and transforms the current DNA micro array technology into a high throughput screening tool.

In this specific patent application, a process of using the high and medium throughput micro array (generally the “KnowledgeWell™ bio grid array”) is described. A preferred embodiment of the method and system of the present invention may utilize a single label of for example, isotope, colorimetric chemical, fluorescence, metal or other suitable reflective compound in parallel processing of multiple samples, for example, 6, 24, 96, 384, 1536 or more samples at the same time on the same platform, as opposed to the current single sample plus a control as limited by the microscope slide format.

It is an objective of this invention to utilize DNA micro array chip technology to profile multiple gene expression patterns in the context of a living cell. Such gene expression profiling will enable scientists to gain specificity and selectivity in the process of screening a potential drug lead. The current patent application describes a process and system to utilize the innovative KnowledgeWell™ bio grid array platform technology as described in Applicant's prior application to process high volumes of samples. The readout of such process will result in differential gene expression profiles. Specifically, one embodiment of the present patent application covers a process using one label, isotopic, colorimetrical, fluorescent, or reflective material such as a metal to do parallel analysis of multiple samples of differential gene expression.

In particular, the current patent application defines a process that relies on a single label: isotopic, colorimetric, fluorescent label, or metal or other reflective material. The singularly labeled sample is preferably processed in specific formats of a modified microtiter plate in 6, 24, 96, 384, or 1536 well formats. The differential comparison of two-sample analysis using the current microscope slide format DNA micro array is replaced with a parallel processing of multiple samples using the microtiter plate format high and medium throughput bio grid array.

The invention integrates processes of the current genomics DNA micro array and the high throughput drug screening technologies.

Thus, it is an aspect of the invention to provide a DNA micro array assay that requires only one label for differential gene expression analysis.

It is another aspect of the invention to provide a single label DNA micro array assay for analyzing differential gene expression that can be done in microtiter plate format.

It is a further aspect of the invention to provide a DNA micro array assay that can be done more cost effectively and quickly than current two-label, microscope slide format DNA micro array assays.

It is yet another aspect of the invention to provide a single label DNA micro array assay for differential gene expression analysis that can be performed in a high throughput screening format.

It is a further aspect of the invention to provide a DNA micro array assay that may be done in parallel for multiple samples, with single or multiple labeling of samples.

A still further aspect of the invention is to provide a microtiter plate format for DNA micro array assay. Such microtiter plate format was described in Applicant's previous patent application as a glass and plastic hybrid plate.

These and other advantages of the present system will become apparent upon examination of the accompanying Figures and detailed description of the invention.

### BRIEF DESCRIPTION OF THE DRAWINGS

FIG. 1 illustrates the current manual sample processing method using two labels for differential gene expression analysis using the current DNA micro array.

FIG. 2 illustrates the single label process of the present invention as performed in a microtiter plate format.

### DETAILED DESCRIPTION OF THE INVENTION

The present invention defines a process, method and system that may utilize one, instead of the current two, labels of an isotopic, colorimetric, fluorescent, or reflective material to process multiple samples in parallel. The process of multiple sample comparison is achieved in applicant's SBS microtiter plate format using the high and medium throughput “bio grid” array previously described in Applicant's prior related application.

Unless defined otherwise (such as with Applicant's previously filed KnowledgeWell™ bio grid array microtiter plate micro array assay platform), all technical and scientific terms used herein have the same meaning as commonly understood by one of ordinary skill in the art to which this invention belongs. Generally, the nomenclature used herein and the laboratory procedures in spectroscopy, drug discovery, cell culture, molecular genetics, plastic manufacture, polymer chemistry, diagnostics, and amino acid and nucleic acid chemistry described below are those well known and commonly employed in the art. Standard techniques may be used for preparation of plastics, signal detection, recombinant nucleic acid methods, polynucleotide synthesis, and microbial culture and transformation. Fluorescent labeling techniques and procedures are generally performed according to conventional methods in the art for fluorescence techniques. Many standard techniques are used for chemical syntheses, chemical analyses, biological, and micro array assays.

“Dye” refers to a molecule or part of a compound that absorbs specific frequencies of light, including but not limited to ultraviolet light.

“Label” or “labeled” refers to incorporation of a detectable marker, e.g. by incorporation of a radioactive, fluorescent, colorimetric or other moiety that can be detected. Various methods of labeling polypeptides, nucleotides, DNA, RNA and other biological molecules are known in the art and may be used.

“Plate” refers to a multi-well microtiter type plate, unless otherwise modified in the context of its usage, for example the modified microtiter plate format “KnowledgeWell™ micro array platform of Applicant's prior application, of which this Application is a Continuation-in-Part.

“DNA chips” refers to high-density oligonucleotide arrays generated by in-situ methods.

“DNA micro arrays” refers to low and medium density cDNA or oligonucleotide arrays generated by microspotting DNA samples onto glass.

“Slide” refers to the standard DNA micro array microscope slide substrate on which DNA micro array assays are conventionally performed.

“KnowledgeWell™ bio grid array” refers to the microtiter plate formatted micro array platform of Applicant's prior Application, and to the micro arrays spotted thereon. Arrays comprised of various types of material would be referred to by the type of material in the array, for example: “Bio Grid Array” more generally, but also “Gene Grid Array”, “Protein Grid Array”, “PNA Grid Array”, “Cell Grid Array” or any other material that would be contained in an array. Applicant's microtiter plate formatted micro array platform may be, for example, in the footprint of a 96, 384 or 1536 well microtiter plate. The base, as described in Applicant's prior application is a glass material and is overlaid with a bottomless plastic material in a grid having wells divided therein and being adhesively bound to the glass base to create wells with plastic walls and glass bottoms. The wells of a 96 well platform are typically at 9.0 mm well to well spacing (or “pitch center”), the wells of a 384 well platform are typically at 4.5 mm well to well spacing, and, as a final example, the wells of a 1536 well platform are typically at 2.25 mm well to well spacing. Platforms having other numbers of wells and spacing are also possible, including as few as 6 wells. The platform, briefly described above, in which the assay described below is performed, is described in detail in Applicant's prior application.

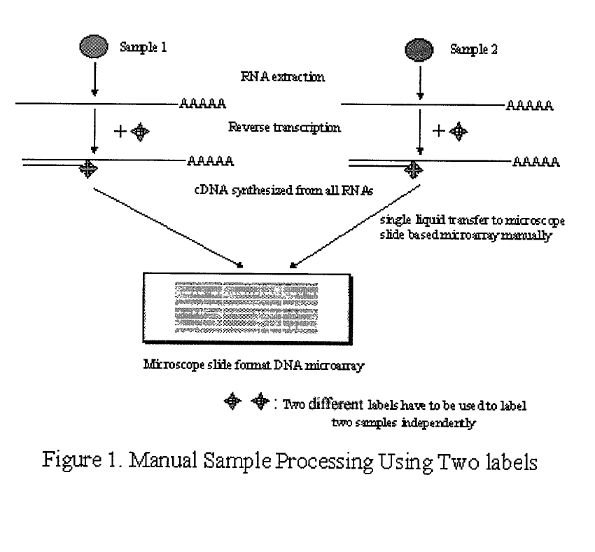

Currently, differential gene expression analysis is performed as shown in FIG. 1, using two labels, one on each of two samples, to compare the two samples on a microscope slide, with an array size of about 1×1 to 2×2 cm square area. For example, the most basic experiment requires a control as one sample and a test sample as the second sample on the same slide, with the idea being to compare the two and identify the differences. When using the traditional microscope slide format for DNA micro array assays, both the control and the test sample are labeled with unique, usually fluorescent, dyes which are excitable at different wavelengths. Both the control and the test sample can be exposed to the same micro array at the same time on one microscope slide.

As described above, the labeled samples are deposited on a microscope slide on which is immobilized an array of various genes or portions of genes, and are allowed to hybridize to the immobilized material. The amount of gene expression of each of the two samples is measured by the amount of signal generated by the labels on the samples bound to the immobilized material. After the hybridization, the microscope slide is read in a scanner with two color filters. Each filter corresponds to the excitation frequency of the respective control or test sample fluorescent labels. The number and strength of the signals is then analyzed to determine the differential gene expression of the samples.

The reason current DNA micro array assays require two labels is to facilitate assaying two samples at the same time on the same array, or (in other words) on the same chip, and to provide a control. In reality only one test sample may be assayed per chip because the other sample must be a control. There was an advantage to having two different labels representing two different but related samples to compare by hybridizing them to the same set of DNA's (genes or portions thereof) on one chip. In this approach, the control is used as an internal control to the array itself since the DNA “spots” (the immobilized material making up the array) to which the control and test sample are hybridized are the same for both the control and the test sample.

As spotting technology has become more mature, the DNA spots on a chip have become much more consistent and uniform. In other words, the arraying technology has become more reliable. However, with the present technology two labels are still required. Using two labels largely constrains researchers to comparing no more than two samples (or really one sample vs. a control) in any given experiment—i.e. on any given chip or microscope slide. In addition the size or dimension of the arrays, and therefore the total number of spots per slide is limited by the size of the microscope slide. Thus, cost plays a major role in DNA micro array assaying because many, many microscope slides must be used, and many arrays deposited, or spotted, individually in order to assay large numbers of samples.

Since differential gene expression techniques are used to compare differences of gene expression of different samples, it is important to keep experimental conditions and chemistry as consistent as possible. Commonly reported problems include sample variations due to differences in dye structure, dye incorporating efficiency, optimal labeling conditions, etc. In addition, as noted above, the number of samples that can be analyzed at one time is limited by the microscope slide format.

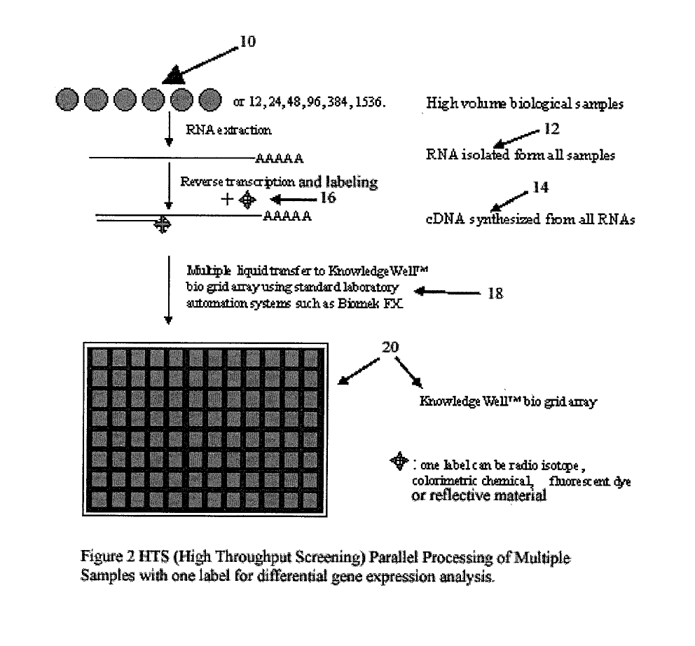

Referring now to FIG. 2 of the present invention, in which like reference numerals refer to like elements throughout, a most basic embodiment of the invention includes a set of biological samples 10, preferably at least 6 but as many as about 1536 or more samples. The samples may be, for example, various stages of a particular cancer cell treated with millions of different chemical entities, or for example blood samples of tens of thousands of clinical trial participants. RNA is then extracted and 12 from each sample and mRMA is isolated and used as a template for cDNA synthesis 14 in the presence of one label 16. Using only one label 16 provides advantages such as providing uniform labeling conditions and chemistry of one dye, instead of trying to label with two or more dyes. Thus, using just one label, instead of two or more, enables users to obtain much more consistent labeling conditions. The label 16 may be a radiolabel, a colorimetric label, a fluorescent label, a metal, metallic or other reflective label, or any other suitable label that can be incorporated by the samples and that will result in a detectable change or signal. For example, a heavy metal can be used to label DNA and read by scanning any changes in reflection caused by the heavy metal label.

The labeled cDNA samples are then deposited 18 in to the KnowledgeWell™ bio grid array microtiter plate format assay platform 20, on the bottom inner glass surface of each well of which is immobilized an array of various known reagents including genes or portions thereof including DNA or RNA sequences, PNA, polynucleotides or polypeptide biopolymers such as known DNA fragments, cDNA or short oligonucleotides, proteins or polypeptides. As noted above, the various types of materials from which arrays for the present invention may be made would give their names to the arrays, for example “PNA Grid Array” etc. The labeled samples, once deposited in the wells, are then allowed to hybridize with the various immobilized reagents, and any resulting binding is measured by reading the signals from the label in a reader selected based on the label. Analysis is then performed from the various signals using software that acquires and processes the data into information useful for analysis.

The depositing of sample into the microtiter plate format platform, on which the known reagents have already been deposited and immobilized in an array, may be performed using standard laboratory automation systems such as the Beckman Coulter Biomek FX, Tecan Genesis, or Zymark Sciclone ALH liquid handlers. It is possible to use standard automation systems because the KnowledgeWell™ bio grid array, microtiter plate formatted micro array assay platform (described in Applicant's prior application) conforms to the SBS standards described above and has the footprint of a standard microtiter plate.

However, with the present invention many more samples may be assayed at once because an array, of for example, an approximately 9 mm×9 mm array for a 96 well platform, an approximately 4.5 mm×4.5 mm array for a 384 well platform or an approximately 2.25 mm×2.25 mm array for a 1536 well platform may be deposited in each well of the platform, as opposed to the assay being limited to a single array per 1-2 cm square area when the platform is a microscope slide format.

With the present invention, the various dimensions of array sizes are simply the approximate maximum sizes of the arrays, not the densities (number of spots) of each array. The sizes are approximate because the area of the bottom of each well is approximate and the arrays cannot extend right up to the wall of the well, due to limitations in spotting/deposition technology and difficulty in reading if the array spots are right up against the wall of the well. Thus 9.0×9.0 mm, 4.5×4.5 mm and 2.25×2.25 mm are the approximate maximum sizes of the arrays. Other size arrays are possible as well, including arrays smaller than the maximum, and arrays of different dimensions in differently sized wells, for example the arrays of a 6 well plate would be based on the area of the bottom of each of the 6 wells which is about 36 mm×36 mm. Similarly the approximate area of the bottom glass surface of each well of a 24 well plate would be about 18 mm×18 mm. Thus the range of approximate maximum array sizes from a 6 well platform to a 1536 well platform is 36 mm×36 mm down to 2.25 mm×2.25 mm.

The deposited arrays may be smaller in area than the maximums for each well number and size, depending on the density of the array. As noted above the given maximum area dimensions for the arrays are the approximate bottom surface area of the wells, based on the conformation of Applicant's hybrid plates to the SBS standards for microtiter plates, including overall footprint dimensions and well sizes and spacings. The diameter, depth, and volume etc. of the wells are not referred by the maximum array dimensions. The array dimensions given disclose the approximate maximum area on the glass inner bottom surface of each well that is covered by the array.

In addition, although the maximum area possible to be covered by each array in each different platform (6, 24, 96, 384, 1536 wells etc.) remains the same, arrays of various densities, and thus various sizes, may be deposited in the same size wells. Also, as described above, the area covered by each array would be less than and up to about equal to the maximum array areas given above for wells of each platform. For example, a 75×75 array or a 50×50 array could be deposited in a 9.0 mm×9.0 mm area in a 96 well platform. The 50×50 array would have the same spot to spot spacing but would just cover a smaller area. While it is preferable to use the same spot to spot spacing for all arrays (and thus the area occupied by arrays of different density would vary), it is also possible to spot arrays at varying spot to spot spacings in order to have each array occupy the same area (per same number of well platform), independent of the density of the arrays. However, varying the spot to spot spacing (in order to keep the area occupied by each array constant) makes reading extremely difficult because the reader would have to be adjusted to read spots at different spacings or locations (if the spot locations were moved the reader might not be able take proper readings). Reading is much easier if the spot to spot spacing of each array is kept constant no matter what the density of the array. The size or area occupied by the arrays would then vary but reading would be much easier because the reader would not have to be adjusted for varied spot spacing.

Finally, it is also possible to have “non-square” arrays, as long as their dimensions are no greater than the maximum optimal array area for a given platform. By “non-square” Applicant means an array wherein the number of columns differs from the number of rows, for example a 65×75 array (non-square) as opposed to a “square” 75×75 array. As noted above it is possible to adjust the spot spacing to make every array physically square in shape, but it is not necessary. Spot spacing is preferably kept constant and “non-square” arrays would simply be rectangular in shape within the maximum preferred area of each well for the number of wells in the platform being used. See Applicant's prior application for the minimum and maximum preferred array densities for use in each of several platforms, including 96, 384 and 1536 well platforms.

Therefore, using Applicant's high and medium throughput bio grid array one can achieve parallel comparison of multiple samples up to millions of samples. Using the microtiter plate formatted micro array platform and more modern, mature arraying technology enables a micro array to be accurately and consistently deposited simultaneously in each well of the microtiter plate platform if desired. This provides maximal reliability in spotting and enables the comparison of as little as about 6 samples up to 96, 384, or 1536 samples on one single platform, depending on how many wells the platform has and how many wells are used. As noted above, with the current microscope slide technique for example, a comparison of 96 samples vs. controls would require 96 chips or slides. Thus it can be seen that the present invention, using only a single label can process many more samples simultaneously than is possible using an array on a microscope slide.

The description below further illustrates the parallel processing ability of the present invention. With the present invention, as few as preferably about 6 up to about 1536 samples may be processed in parallel, i.e. simultaneously on the same platform depending on the number of wells in the platform and how many wells are used. For example, in each well of a 96-well microtiter plate formatted platform there may be an array of 500 different genes or other known reagents. All 96 wells may have an identical approximately 9.0 mm×9.0 mm square array of the same 500 genes all deposited uniformly at the same time. Thus, 96 different samples may be studied with respect to the 500 genes all at the same time.

A control could be used in one well, and would have the same label as the other 95 samples but would be in its own well. Or, if desired, for example with a 96 well format, a column of 8 wells could be used as a dose curve of the control while the remaining 88 wells would be used for samples. If multiple plates were prepared and assayed simultaneously, a single control or dose curve of control need only be used on one of the plates, or on for example every 5^{th}, 10^{th} or 20^{th} etc. plate, as desired by the user. Also, for additional confidence, more than one control may be used, either in a separate single well, or a separate column with a dose curve of the control.

A “universal” control sequence, such as those universal control sequences known and used in the art, or any newly developed and adopted universal control, could be used with the present invention. The universal control could be any universal control sequence including insect, viral, and bacterial DNA or RNA. Thus, using a universal control for each microtiter plate platform provides an even greater intra-well normalization tool, as well as an inter-well normalization tool. The universal control sequence, when labeled, can be used to define background, and to align image in the reader or scanner. Thus, Applicant's method provides the ability to have multiple samples in separate wells that are all processed (deposited, hybridized, and read) simultaneously using a control, including conventional universal control sequences, as normalization tools.

The method of the invention allows multiple samples to be assayed in the format of a dose response curve, a time course, a native vs. agonist vs. antagonist format, or any other type of assay that can be used for gene expression profiling, and it also allows comparison of multiple cell lines at the same time under uniform conditions.

With the current technology each of the samples would have to be assayed on a separate slide, each with the control sample and using two labels. 96 separate microscope slide arrays would have to be made. Thus, the present invention provides both time and cost savings by enabling deposition of almost any number of arrays simultaneously and studies of thousands or millions of samples simultaneously.

In addition, therefore, with the present invention, only one type of scanner or reader is required because only one label is required. Each sample is processed separately in its own well; so all samples can have the same label. Furthermore, with the method and system of the present invention, as noted above, well-to-well variation can be normalized for the background and thereby produce accurate intra- and inter-well control.

In the preferred embodiments, the label incorporated into the cDNA samples may be for example: a radioisotope, a colorimetric chemical, a fluorescent dye, or a metal (with which changes in light reflection would be detected). Comparison is then made to profile respective gene expression levels amongst all the samples using only the one label. For each gene, or spot, in each array in Applicant's KnowledgeWell™ bio grid platform, the expression level can be compared amongst all samples using only one label.

Thus, an additional advantage of the present invention is that the single label is incorporated into the sample's cDNAs under one uniform set of chemical conditions. Such uniform conditions can eliminate common sample variations due to differences in dye structure, incorporating efficiency, optimal labeling conditions, etc.—these types of variations have been widely reported in the literature as problematic but are overcome by the present invention.

However, the above embodiment of a parallel processing method and system of the present invention is not limited to a single label. While there is no need for two labels because there may be one sample per well, samples can also be “multiplexed” using two or more labels. In this embodiment more than one sample can be analyzed per well, and thus approximately twice as many samples (or more) may be assayed in one microtiter plate platform using two (or more) labels vs. using only one label. For example, two samples, with two different labels—for example two different fluorescent dyes—may be deposited in the same well, similarly to the way they would be used on the same microscope slide, and a multi-color scanner would be used as with traditional micro arrays. Thus samples numbering at least two or more times the number of wells of a given platform may be analyzed on that given platform simultaneously using multiple labels.

With this embodiment, as with the single label, a single control for the whole microtiter plate platform could be used, either in a single well or a series of wells, and the remaining wells could each contain two test samples. Thus, the control, which may be any suitable control, such as known universal control sequences, would provide a normalization tool as with the single label embodiment but approximately twice as many samples could be assayed per platform. Thus, although there would be increased labeling time and variation, and increased sample deposition time there would still be substantial time and cost savings vs. microscope slide assays because many times the number of samples could be analyzed on one microtiter plate platform vs. one microscope slide.

Thus the present method and system of depositing a micro array into each well (or any desired number of wells—every well need not be used) of Applicant's multi-well microtiter plate micro array platform enables simultaneous parallel single or multiple label processing of many more samples than is possible with traditional microscope slide format micro arrays which require two labels and which are limited in size to a total area of 1×1 to 2×2 cm square. While the individual area occupied by each array of Applicant's invention is much smaller than an array 1-2 cm square, for example 9.0 mm×9.0 mm, the total number of samples processed per platform is much greater than that possible with a 1-2 cm square microscope slide.

The present invention therefore provides solutions to the automation, procedural, accuracy, reproducibility, and cost shortcomings of the current DNA micro array assay in addition to providing a solution to the physical limitations of the microscope slide format. The present invention, in combination with Applicant's KnowledgeWell™ bio grid platform and arrays deposited therein (See Applicant's prior application) provides a process and system that enables parallel comparison of multiple samples, up to millions, using currently available lab automation equipment. The invention may be used with only a single label if desired, but may also be used with two or more labels. A major advantage of the present invention is the ability to process multiple samples simultaneously in a microtiter plate format using already installed and available laboratory automation. Only the method and system of the present invention allows for easy experimental designs for dose response curves, time point analysis, multiple treatment types, agonist vs. antagonist at different concentrations, etc. It is simply not practical with current micro array technology to process the equivalent number of microscope slide format assays as would be required for the above described experiments or studies. Even with much smaller array sizes, many more samples are able to be processed with the system and method of the present invention than is possible with the current microscope slide format because the platform of the present invention as a whole is much larger than 1-2 cm square and can hold many more array spots than the current 1-2 cm square area arrays. Thus, the present invention greatly increases the number of micro array assays that can be performed at once, on the same platform, and greatly simplifies the preparation and performance of the assays while improving accuracy. It also eliminates many of the manual preparation and performance steps of the current assays, because the microtiter plate formatted platform of the present invention is compatible with current laboratory automation instrument systems.

While the invention has been described with reference to the preferred embodiment, the foregoing description is illustrative only. Those of ordinary skill in the art will see that the specific embodiments described are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawing figures. Such modifications are intended to fall within the scope of the appended claims.

What is claimed is:

1. A method and system for simultaneous analysis of samples, comprising:
obtaining a number of samples to be analyzed;
extracting RNA from each said sample to be analyzed;
isolating mRNA from said RNA to use as a template for synthesizing DNA;
synthesizing cDNA from each said mRNA of each said sample;
labeling each said cDNA with a label;
depositing an array of known reagents into as many wells of a multi-well microtiter plate platform as desired for a particular assay and immobilizing each said array thereon;
depositing at least one of said labeled cDNA into at least one well of said multi-well microtiter plate platform;
depositing at least one said labeled cDNA into as many said wells having a said array therein as desired for a particular assay;
allowing said each said labeled cDNA to hybridize to said array of known reagents in each said well;
reading said microtiter plate platform after hybridization is completed; and
using software, processing signals generated and read from said at least one label into a format useful for analysis.

2. The method according to claim 1 wherein said number of samples to be assayed simultaneously is at least about 6.

3. The method according to claim 1 wherein said samples are chosen from the group consisting of: DNA, RNA, PNA, genes, portions of genes, polynucleotides, polypeptide biopolymers, fragments of DNA, fragments of RNA, short oligonucleotides, proteins and polypeptides.

4. The method according to claim 1 wherein said label is chosen from the group consisting of: a fluorescent label, a radio label, a colorimetric label, or a reflective label.

5. The method according to claim 4 wherein said reading is performed on a device capable of reading a signal chosen from the group consisting of:
fluorescence, radioactivity, color intensity, and reflection changes.

6. The method of claim 1 wherein two samples are deposited in each well of said microtiter plate platform.

7. The method of claim 6 wherein each of said two samples is labeled with a different label.

8. The method of claim 7 wherein said reading is performed using a device capable of simultaneously reading two of the same type of signals.

9. A method and system of parallel analysis of samples simultaneously, comprising:
obtaining a number of samples to be analyzed;
extracting RNA from each said sample to be analyzed;
isolating mRNA from said RNA to use as a template for synthesizing DNA;
synthesizing cDNA from each said mRNA of each said sample;
labeling each said cDNA with a label;
depositing an array of known reagents into as many wells of a multi-well microtiter plate platform as desired for a particular assay and immobilizing each said array thereon;
depositing one of said labeled cDNA into a well of said multi-well microtiter plate platform;
depositing one said labeled cDNA into as many said wells having a said array therein as desired for a particular assay;
allowing said each said labeled cDNA to hybridize to said array of known reagents;
reading said microtiter plate platform after hybridization is completed; and
using software, processing signals generated and read from said label into a format useful for analysis.

10. The method according to claim 9 wherein said number of samples to be assayed simultaneously is at least about 6.

11. The method according to claim 9 wherein said samples are chosen from the group consisting of: DNA, RNA, PNA, genes, portions of genes, polynucleotides, polypeptide biopolymers, fragments of DNA, fragments of RNA, short oligonucleotides, proteins and polypeptides.

12. The method according to claim 9 wherein said label is chosen from the group consisting of: a fluorescent label, a radio label, a colorimetric label, and a reflective label.

13. The method according to claim 12 wherein said reading is performed on a device capable of reading a signal chosen from the group consisting of:
fluorescence, radioactivity, color intensity and reflection changes.

14. The method according to claim 9 wherein each said sample is labeled with the same said label.

15. A method for multiple parallel analysis of samples simultaneously, comprising:
obtaining a number of samples to be analyzed;
extracting RNA from each said sample to be analyzed;
isolating mRNA from said RNA to use as a template for synthesizing DNA;
synthesizing cDNA from each said mRNA of each said sample;
labeling each said cDNA with one of either a first or a second label;
depositing an array of known reagents into as many wells of a multi-well microtiter plate platform as desired for a particular assay and immobilizing each said array thereon;
depositing one said cDNA labeled with said first label, and one said cDNA labeled with said second label into the same well of said multi-well microtiter plate platform;
depositing both a said cDNA labeled with said first label and a said cDNA labeled with said second label in as many wells having a said array therein as desired for a particular assay;
allowing said both said labeled cDNAs to hybridize to said array of known reagents in each said well;
reading said microtiter plate platform after hybridization is completed; and
using software, processing signals generated and read from said first and said second labels into a format useful for analysis.

16. The method according to claim 15 wherein said number of samples to be assayed simultaneously is at least about 6.

17. The method according to claim 15 wherein said samples are chosen from the group consisting of: DNA, RNA, PNA, genes, portions of genes, polynucleotides, polypeptide biopolymers, fragments of DNA, fragments of RNA, short oligonucleotides, proteins and polypeptides.

18. The method according to claim 15 wherein said first and said second labels are chosen from the group consisting of: a fluorescent label, a radio label, a colorimetric label, and a reflective label.

19. The method according to claim 15 wherein said reading is performed on a device capable of reading simultaneously two of the same type of signals chosen from the group consisting of: fluorescence, radioactivity, color intensity, and reflection changes.

20. The method according to claim 1 wherein a universal or other control sample is deposited in at least one well of said microtiter plate platform, for use as an intra-well and inter-well normalization tool, to define background and align image for reading said microtiter plate platform.

21. The method according to claim 9 wherein a universal or other control sample is deposited in at least one well of said microtiter plate platform, for use as an intra-well and inter-well normalization tool, and to define background and align image for reading said microtiter plate platform.

22. The method according to claim 15 wherein a universal or other control sample is deposited in at least one well of said microtiter plate platform, for use as an intra-well and inter-well normalization tool and to define background and align image for reading said microtiter plate platform.

23. The method of claim 1 wherein said array of known reagents is deposited on the inner bottom surface of said well on an area in the maximum range of about 2.25 mm×2.25 mm to about 36.0 mm×36.0 mm, said area being dependent upon the number and size of wells formed in said microtiter plate platform and the density of the array deposited therein.

24. The method of claim 9 wherein said array of known reagents is deposited on the inner bottom surface of said well on an area in the maximum range of about 2.25 mm×2.25 mm to about 36.0 mm×36.0 mm, said area being dependent upon the number and size of wells formed in said microtiter plate platform and the density of the array deposited therein.

25. The method of claim 15 wherein said array of known reagents is deposited on the inner bottom surface of said well on an area in the maximum range of about 2.25 mm×2.25 mm to about 36.0 mm×36.0 mm, said area being dependent upon the number and size of wells formed in said microtiter plate platform and the density of the array deposited therein.

26. The method of claim 23 wherein said inner bottom surface of each said well is glass.

27. The method of claim 24 wherein said inner bottom surface of each said well is glass.

28. The method of claim 25 wherein said inner bottom surface of each said well is glass.

29. A method and system for multiple parallel analysis of samples simultaneously, comprising:
depositing an array of known reagents into as many wells of a multi-well microtiter plate platform as desired for a particular assay and immobilizing each said array thereon;
depositing at least one labeled cDNA into at least one well of said multi-well microtiter plate platform;
depositing at least one said labeled cDNA into as many wells having a said array therein as desired for a particular assay;
allowing said each said labeled cDNA to hybridize to said array of known reagents in each said well;
reading said microtiter plate platform after hybridization is completed; and
using software, processing signals generated and read from said at least one label into a format useful for analysis.
